(19) United States
(12) Patent Application Publication
(10) Pub. No.: US 2007/0282360 A1
(43) Pub. Date: Dec. 6, 2007

(54) Tongue scraper

(76) Inventors: Kimberly H. Smith, Tulsa, OK (US); Loren Smith, Stillwater, OK (US)

Correspondence Address: SUNG I. OH, PROFESSIONAL LAW CORPORATION, 710 QUAIL VALLEY LANE, WEST COVINA, CA, 91791, US

(21) Appl. No.: 11/444,231

(22) Filed: May 30, 2006

(51) Int. Cl. A61B 17/24

(52) U.S. Cl. 606/161

(57) ABSTRACT

A tongue cleaning device includes a sponge-like tip that is fluidically connected to a refillable reservoir that will contain mouthwash or the like. A scraper is located adjacent to the tip and a check valve in the reservoir prevents fluid from flowing away from the tip in the event the orientation of the device is suddenly changed.

### TECHNICAL FIELD OF THE INVENTION

The present invention relates to a device for personal cleaning and, more particularly, to a device for removing undesired matter from a user's tongue and, even more particularly, the invention relates to a tongue-treating device for removing plaque and debris from the tongue in order to prevent halitosis and for applying a treating substance onto the tongue.

### BACKGROUND OF THE INVENTION

Halitosis is a concern for many people affected of this disorder that is chronic or pathological disorder stemming from non-oral sources as well as oral sources. Studies have shown that about 85% patients suffering from halitosis have an oral condition as the source. Measures of oral malodor suggest that it is caused mainly by tongue coating as the result of anaerobic sulfur producing bacteria, which normally live within the tongue. This bacteria is supposed to be there because they assist digestion by breaking down proteins found in specific foods, mucus, blood, and death cells. Under certain conditions, they start to break down the proteins found in those specific foods.

Tongue coating comprises desquamated epithelial cells, blood cells, bacteria and mucus. The morphology of the dorsal surface of the tongue is very irregular with the presence of multiple fissures and filiform and fungiform papillae. These fissures and crypts may create a unique ecological site were microorganisms are well-protected from the flushing action of the saliva and where oxygen levels are low enhancing the growth of anaerobic bacteria. Saliva from nearby glands drips down on the posterior region of the tongue, which is full of irregularities. The bacteria on the tongue are also a potential contributor to periodontal disease and other oral health problems. This coating on the tongue also causes a loss of taste since it dulls the taste receptors. Furthermore, since the human tongue has numerous protuberances, e.g., papillae, which convey to the brain the senses of taste and touch, the tongue can easily become a breeding ground for microorganisms such as bacteria, as well as a repository for food debris, volatile sulfur compounds (which are a major cause of halitosis) and dead cells. Over time, some of the collected material becomes a soft plaque which is another cause of bad breath. This soft plaque is known to attack the teeth and gums.

Still further, the anaerobic bacteria break down specific components of the coating of the tongue creating certain gases or volatile sulfur compounds (VSCs). These VSCs have been implicated as a major contributing factor to halitosis. Consequently, the removal of the tongue coating reduces VSCs production and longer lasting reductions in VSC levels are followed after tongue scraping. Methods that involved cleansing of the dorsoposterior surface of the tongue caused the most pronounced reductions of halitosis.

Tests have shown that daily scraping to reduce the amount of coating on the tongue eliminates much of the undesirable bacteria and sulfur compounds, thus significantly inhibiting plaque formation on the teeth in the long term, and substantially reducing halitosis in the short term. However, the surface cells of the tongue are sensitive to injury. Therefore, the removal of the bacteria and waste material should be performed in a manner which respects the sensitivity of the tongue.

While the inventor is aware of several devices that can be used to scrape the tongue, the inventor is not aware of any tongue scraping device that will not only scrape the tongue in a manner which respects the sensitivity of the tongue but will also provide a means for cleansing and disinfecting the tongue.

### SUMMARY OF THE INVENTION

The above-discussed disadvantages of the prior art are overcome by a tongue cleaning device that includes a sponge-like tip that is fluidically connected to a refillable reservoir that will contain mouthwash or the like. A scraper is located adjacent to the tip and a check valve in the reservoir prevents fluid from flowing away from the tip in the event the orientation of the device is suddenly changed.

Using the tongue cleaning device embodying the present invention will permit a user to clean his or her tongue, especially in the back, using a soft sponge that can be coated with liquid that will not only clean the tongue but will serve as a mouthwash. Once the sponge cleaning is completed, the user can simply flip the device over and scrape off the tongue.

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the following claims.

### BRIEF DESCRIPTION OF THE DRAWING FIGURES

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a front elevational view of a tongue cleaning device embodying the present invention.

FIG. 2 is a rear elevational view of the tongue cleaning device shown in FIG. 1.

FIG. 3 is a side elevational view of the tongue cleaning device shown in FIG. 1.

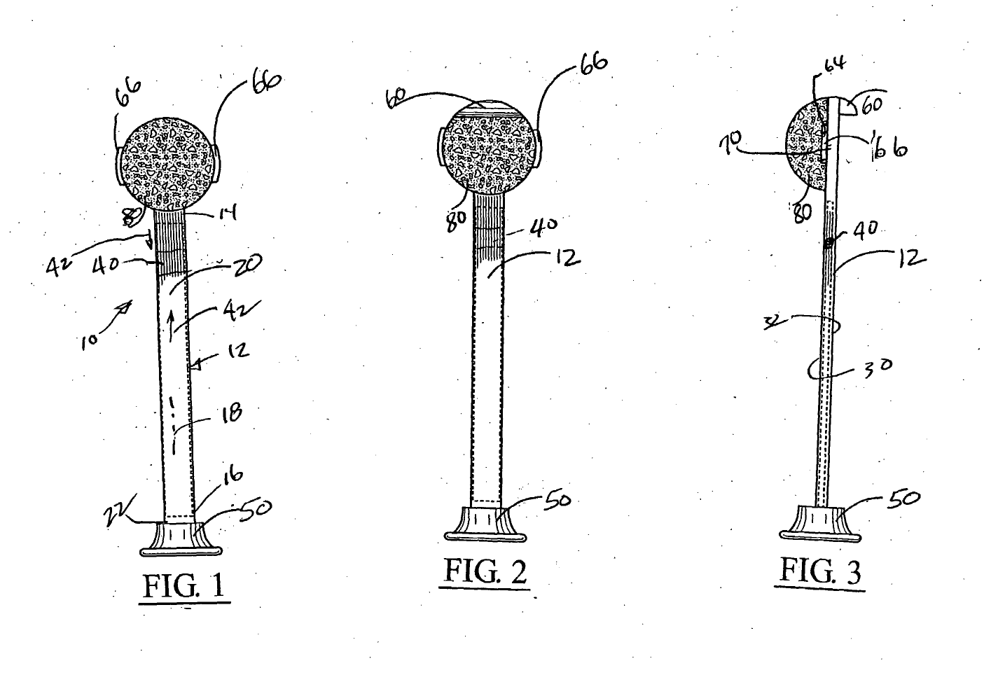

### DETAILED DESCRIPTION OF THE INVENTION

Referring to the figures, it can be understood that the present invention is embodied in a tongue cleaning device 10 which will permit the cleaning and scraping of a user's tongue as well as the dispensing of liquid such as mouthwash which will further enhance the cleaning function. Device 10 comprises a handle 12 which has a first end 14 which is a forward end when the handle is in use, a second end 16 which is a rear end when the handle is in use and a longitudinal axis 18 which extends between first end 14 and second end 16 of the handle.

Handle 12 is hollow and has a fluid-accommodating chamber 20 defined therein. Liquid, such as mouthwash or the like is contained in chamber 20. Screw threads 22 are located adjacent to second end 16. The first and second ends of the handle are open to the chamber so fluid can be placed in the chamber and can flow out of the chamber as will be understood from the teaching of this disclosure. The chamber is re-fillable via the second end of the handle. Handle 12 further includes a first surface 30 and a second surface 32. Handle 12 is elongate and is in the shape of a toothbrush handle in the form shown in the figures.

A one-way check valve 40 is located in fluid-accommodating chamber 20 near first end 14 of the handle. The check valve is oriented in the handle to permit fluid located in the fluid-accommodating chamber to flow in direction 42 toward the first end of the handle and to prevent fluid located in the fluid-accommodating chamber from flowing in direction 44 from first end 14 toward the second end of the handle.

A base 50 is threadably mounted on the handle at the second end of the handle and closes the fluid-accommodating chamber at the second end of the handle when the base is in place on the handle. The base can be used to stand device 10 in an upright orientation as shown in the figures. This will enable the device to be stored and used in an efficient manner.

A tongue scraper element 60 is located on second surface 32 of the handle adjacent to first end 14 of the handle. The tongue scraper is used in the manner known to those skilled in the art to scrape the user's tongue, especially near the back of the tongue. A sponge-mounting bracket 64 is mounted on first surface 30 of the handle adjacent to first end 14. Further sponge mounting elements, such as brackets 66, are also located on the handle.

A fluid port 70 is defined through first surface 30 of the handle and through sponge-mounting bracket 64. Fluid port 70 is in fluid communication with fluid-accommodating chamber 20 via check valve 40. Fluid in chamber 20 will flow in direction 42 toward port 70 but will not flow in direction 42 away from port 70 so that if the device is tipped during use, fluid will not flow away from port 70.

A fluid-dispensing sponge 80 is releasably mounted on first surface 30 of the handle by sponge-mounting bracket 64 and brackets 66. The sponge is in fluid communication with fluid-accommodating chamber 20 via fluid port 70. The sponge is replaceably mounted on the handle so it can be used and then replaced as often as necessary.

Use of device 10 can be understood from the teaching of the foregoing disclosure and thus will only be briefly discussed. A user stores device 10 on the base 50 with fluid in chamber 20. The device is used by washing a user's tongue with the sponge which will also apply fluid to the tongue. The device can also be used to scrape the user's tongue by scraping scraper 60 over the tongue. Fluid can be replaced in chamber 20 as needed.

While various embodiments of the invention have been described, it will be apparent to those of ordinary skill in the art that many more embodiments and implementations are possible within the scope of this invention. Accordingly, the invention is not to be restricted except in light of the attached claims and their equivalents.

What is claimed is:

1. A tongue cleaning device comprising:
A) a handle having
(1) a first end which is a forward end when the handle is in use,
(2) a second end which is a rear end when the handle is in use,
(3) a longitudinal axis which extends between the first end and the second end of the handle,
(4) the handle being hollow and having a fluid-accommodating chamber defined therein,
(5) screw threads located adjacent to the second end,
(6) the first and second ends of the handle being open to the chamber,
(7) a first surface, and
(8) a second surface;
B) a one-way check valve located in the fluid-accommodating chamber near the first end of the handle, the check valve being oriented in the handle to permit fluid located in the fluid-accommodating chamber to flow toward the first end of the handle and to prevent fluid located in the fluid-accommodating chamber from flowing from the first end of the handle toward the second end of the handle;
C) a base which is threadably mounted on the handle at the second end of the handle and closes the fluid-accommodating chamber at the second end of the handle when the base is in place on the handle;
D) a tongue scraper element on the second surface of the handle adjacent to the first end of the handle;
E) a sponge-mounting bracket mounted on the first surface of the handle adjacent to the first end;
F) a fluid port defined through the first surface of the handle and through the sponge-mounting bracket, the fluid port being in fluid communication with the fluid-accommodating chamber via the check valve; and
G) a fluid-dispensing sponge releasably mounted on the first surface of the handle by the sponge-mounting bracket, the sponge being in fluid communication with the fluid-accommodating chamber via the fluid port.

2. A tongue cleaning device comprising:
A) a handle having
(1) a first end which is a forward end when the handle is in use,
(2) a second end which is a rear end when the handle is in use, and
(3) the handle being hollow and having a fluid-accommodating chamber defined therein;
B) a one-way check valve located in the fluid-accommodating chamber;
C) a tongue scraper element on the handle;
D) a fluid port defined through the handle to be in fluid communication with the fluid-accommodating chamber via the check valve; and
E) a fluid-dispensing sponge releasably mounted on the handle, the sponge being in fluid communication with the fluid-accommodating chamber via the fluid port.
